(19) United States
(12) Patent Application Publication
(10) Pub. No.: US 2007/0193586 A1
(43) Pub. Date: Aug. 23, 2007

(54) Multi-purpose face and body condom

(76) Inventor: Grace Elizabeth Vaughn, Bellflower, CA (US)

Correspondence Address: Grace Vaughn, P.O. Box 16031, Long Beach, CA, 90806, US

(21) Appl. No.: 11/358,434

(22) Filed: Feb. 21, 2006

(51) Int. Cl. A61F 6/04

(52) U.S. Cl. 128/844

(57) ABSTRACT

This invention defined the Multi-Purpose Face and Body Condom (MPFBC). Specially designed to encompass the entire penis, as well as the wearer's testicles, and when worn over the mouth, can be inverted, completely encompassing the interior of the mouth, as well as the lips and chin. This condom will provide and effective barrier between partners engaging in sexual intercourse, oral and anal sex would prevent direct body-to-body contact from sexually transmitted diseases, genital warts and pubic lice. A stretchy elastic waistband/headband is integrally attached to either end of shield, which can be a solid band or connecting band with adjustable plastic fastener on waist/headband. To secure testicles from flouncing, a small hole is applied at the base of testicles/chin shield to hook a strong thin rubber support from base of shield between the buttocks onto waistband or waistband adjustable area.

Number 2 is tubular embodiment of condom for giving oral or anal sex. Number 4 is protective shield for face/chin and testicles. Number 6 is the plastic adjustable fastener. Number 8 is the waistband/headband connected/circular. Number 10 is hallowing area on condom for the penis or tongue. Number 12 is hole at base of shield. Number 14 is the durable soft rubber string to hook onto the hole at the base of protective shield, brought between the buttocks and hooks onto the adjustable waistband. Number 16 is the reinforced yet soft rubber hook made on each end of soft rubber string.

### SPECIFICATION

I claim priority to my Provisional Patent, application number US60/700,030 filed Jul. 15, 2005. This is a request for filing a None Provisional Patent based on the disclosed embodiments. Addendum to invention is title change from “All-Purpose Condom” to new title, “Multi-Purpose Face And Body Condom”.

Sex is a healthy and natural part of life. A physical expression of love, commitment or simple passion, a healthy sex life can be invigorating and meaningful. For most sexually active men and women, sex involves the conscious, positive expression of sexual energy in ways that enhance self-esteem, physical health, and an emotional relationship. Mutually beneficial and pleasurable, consensual sex is the cornerstone for most coupled relationships. Because sex can be so physically and emotionally gratifying, a healthy sex life is a key component of most coupled partnerships and a natural part of life for many single people, as well. Unfortunately, having sex is not without risks. Sexually transmitted infections such as Human Immunodeficiency Virus (HIV), syphilis, gonorrhea, Hepatitis B and Human Papilloma Virus (HPV) are rampant in this country, particularly amongst teenagers and young adults. In fact, according to statistics compiled by the Centers for Disease control (CDC), over 15 million cases of sexually transmitted infections are reported in the United States every year, with one in four of these cases being reported by a teenager. Additionally, unwanted pregnancy is a very real consequence of having unprotected sex. Considering that eight out of every ten girls and seven out of every ten boys will engage in sexual intercourse before the age of fifteen, the fact that one million teens become pregnant every year, should hardly seem startling. Although many adults are financially and emotionally capable of handling the consequences of unwanted pregnancy, the risks of pregnancy and STD's are for them, also very real.

Perhaps the best defense against sexually transmitted diseases and unwanted pregnancy is the regular correct use of a condom. Condoms help men and women express themselves sexually and responsibly by greatly lowering the risk of sexually transmitted diseases and unintended pregnancy. Manufactured of latex or synthetic material, condoms are designed to slip over the penis, thereby preventing the exchange of bodily fluids between partners. Inexpensive and readily available, many consumers prefer the condom to other methods of birth control because it can be purchased without a prescription, can be easily carried and is simple to use. Unfortunately, although condoms effectively prevent the exchange of bodily fluids they do not always prevent body-to-body contact. Specifically, during sexual intercourse it is not uncommon for the wearer's testicles to come into direct contact with partner's genitalia, or if engaged in oral sex, their partner's mouth. As difficult as it may seem to discuss, there are many sexually transmitted diseases such as, genital herpes, genital warts and HIV, as well as such maladies as pubic lice, which can be passed between partners, via this direct contact.

Recognizing the potential for a product, which would effectively prevent body-to-body contact during sexual intercourse, oral or anal sex. This unique product invention was developed designated the Multi-Purpose Face And Body Condom (MPFBC). The Multi-Purpose Face and Body Condom is a condom, particularly designed to encompass the entire penis, as well as the wearer's testicles and when inverted, can be worn over the mouth, completely encompassing the interior of the mouth, as well as the lips and chin. In this manner, the Multi-Purpose Face and Body Condom would provide an effective barrier between partners and would prevent direct body-to-body contact. Similar in appearance to a standard condom, the (MPFBC) Multi-Purpose Face and Body Condom would be cylindrical in shape and could be manufactured of lightweight, yet reliable latex or rubber materials, with a solid headband/waistband, or the headband/waistband disconnected with a plastic adjustable fastener. The tubular part of the condom would feature a durable, softly rounded reservoir for use in collecting bodily fluids. As with traditional condoms, the Multi-Purpose Face and Body Condom could feature a reinforced band, positioned at the base of the unit, designed to secure the condom safely in place. The most notable aspect of the Multi-Purpose Face and Body Condom however, and that which sets this unique condom apart from others available on the market, is that the banding which encompasses the base of the condom would extend outwards, creating an ample, circular shaped shield which would completely encompass the wearer's testicles when worn over the penis, as well as the lips and chin when worn in an inverted manner over the mouth. To prevent this circular shield from slipping during use a stretchy elastic waistband/headband is integrally attached to either end of the shield, which can be manufactured either with a solid waistband/headband or band disconnected and fastened with an adjustable plastic fastener to be worn over or around the waist or the back of the head, respectively. The Multi-Purpose Face and Body Condom could be produced in a variety of sizes ranging form small to extra, extra, extra large and etc. Additional considerations could include the manufacture of lubricated or ribbed versions of the MPFBC. The MPFBC could be rolled into a ring shape with the extended safety shield and attached waistband/headband folded neatly for compact storage. For sanitary purposes, this condom would be individually wrapped in a similar manner as standard condoms and could be sold in packages of six units per container.

Use of the Multi-Purpose Face and Body Condom would be very simple and straight-forward. Consumers would simply purchase and use the Multi-Purpose Face and Body Condom in a similar fashion as they would ordinary condoms. When worn during sexual intercourse, the user would step into the circular stretchy elastic waistband and comfortably position the banding about the waist. The user would then roll the MPFBC up over the penis, adjusting the condom so that the tip of the condom was positioned comfortably over the head of the penis so that the circular band located at the end of the condom securely gripped the area at the base of the penis shaft. The wearer would then make sure that the integrated safety shield completely encompassed their testicles and the area directly surrounding the base of the penis, thus effectively preventing direct body-to-body contact during vaginal or anal intercourse. The consumer will also be able to purchase the MPFBC with an adjustable waistband. Another added feature for this condom would be to have a hole made at the base of the condom where the testicles are secured so that a rubber string could hook from the base of the shield between the buttocks to hook to the back of the waistband to keep testicles form flouncing while engaging in sexual intercourse or anal sex.

When engaged in oral sex the wearer (male or female) would utilize the Multi-Purpose Face and Body Condom in the inverted manner. Utilizing the stretchy band as a headband or as an adjustable band, the user would slide the circular band over the head or fasten band on back of the head so that the inverted condom was positioned directly in front of the mouth. The user would then position the attached safety shield so that it completely encompassed their lips and chin. Next, the wearer would allow their partner to slide their penis into the condom. After use, the Multi-Purpose Face and Body Condom would be carefully removed and discarded. As with standard condoms the MPFBC should be handled gently and stored in a cool, dry place.

There are many significant benefits and advantages associated with the Multi-Purpose Face and Body Condom. Foremost, the MPFBC would provide superior protection for those engaged in sexual intercourse. Effectively preventing the transmission of bodily fluids, and proper use of this condom could protect users from unwanted pregnancy. This advantage should prove especially beneficial for unmarried couples and sexually active teenagers. Further, because the Multi-Purpose Face and Body Condom would prevent the transmission of bodily fluids, this practical product could prove a valuable weapon against the spread of sexually transmitted diseases, particularly the HIV virus. Most importantly, the clever inclusion of the shield attachment would prevent the wearer from coming into direct contact with their partner's genitals or mouth. Encompassing the penis and testicles completely when worn during sexual intercourse, and completely encompassing the lips, interior of the mouth and chin when worn during oral sex, this condom would protect the wearer from highly communicable diseases such as HIV, herpes and genital warts, as well as conditions such as pubic lice. Protecting the overall health of the wearer, as well as their partner, the MPFBC would prove invaluable to any sexually active male or female. Encouraging safe conduct, the MPFBC could provide a comfortable alternative to ordinary condoms already on the market. Compact in size, the Multi-Purpose Face and Body Condom would easily and discreetly fit into any purse, wallet or backpack, providing considerable protection whenever needed. Individually packed or sold in sets, the MPFBC would be perfect for home use, as well as for individual sale in public rest room vending machines.

The Multi-Purpose Face and Body Condom is a practical product invention, which would offer consumers ample protection during sexual activity, while enabling them to enjoy optimal sexual pleasure. Lightweight yet durable, use of this cleverly designed prophylactic would prevent the transmission of bodily fluids, while also preventing body-to-body contact, thus enabling the wearer to enjoy safe, worry free sex. Affordably priced, the Multi-Purpose Face and Body Condom should be well received by sexually active male as well as female consumers, an extremely sizable market potential.

Optional, consumers who purchase and use the Multi-Purpose Face and Body condom can use a stocking cap in case of possible hair entanglement, or they can purchase stick-on padded material for waistband/headband if desired.

### BACKGROUND OF THE INVENTION

The Multi-Purpose Face and Body Condom is specially designed to encompass the entire penis, as well as the wearer's testicles and when inverted, can be worn over the mouth completely encompassing the interior of the mouth, as well as the lips and chin as a protective barrier between partners which would prevent direct body-to-body contact against sexually transmitted diseases, genital warts, HIV and other diseases when engaging in sexual intercourse, oral or anal sex.

Bloodsaw, in U.S. Pat. No. 5,409,016 (1995), proposes an oral condom which is a protective mask to protect the facial area from sexually transmitted diseases when engaging in oral, vaginal or anal sex. To keep this oral condom in place, it attaches to the ears.

Johnson, in U.S. Pat. No. 5,016,649 (May 21, 1991), introduces a protective mask made of a tough film material, which wraps over the lower face with a flaccid pouch-like center portion with connecting strings to tie at the back of the head and tie at the back of the neck. This protective mask was invented to protect the wearer against STD's from oral sex or while performing mouth-to-mouth resuscitation.

Hussey, in U.S. Pat. No. 5,582,187 (Dec. 10, 1996), introduces a protective mask made of a thin, flexible shield for the mouth for protection against the transmission of STD's (Sexually Transmitted Diseases) during oral or anal sex. The shield is held with the hand over the mouth or in a holder assembly by interlocking rings to grip the shield to keep the mask against the face of the wearer with looping strings to fit behind the ears to hold the shield in place.

Harding, U.S. Pat. No. 4,949,731 (Aug. 21, 1991), proposes oral prophylactics, which is an elastic and flexible oral prophylactic that conforms to the mouth, and includes impermeable and permeable embodiments.

Esqueda, U.S. Pat. No. 4,974,605 (December 1990), introduces a facial prophylactic mask for the lower face for the prevention of disease. The mask covers the bridge of the nose to under the chin with a breathing passage and mask held on with straps.

The Bloodsaw patent, Johnson's patent, Hussey's patent, Harding's patent and Esqueda's patent all are facial mask's or oral condoms. While all of these condoms possibly do protect the wearer's from contracting transmitted diseases by mouth, these inventions is for the mouth or lower face only. The Multi-Purpose Face and Body Condom has more advantages than these prior related art such as, it can be worn on a male's penis and testicles to prevent body-to-body contact while engaging in sexual intercourse, oral or anal sex. When worn by a woman or man, it can be inverted. These added features make this condom have far more functions than just for the face.

### BRIEF SUMMARY OF THE INVENTION

The Multi-Purpose Face and Body Condom is designed as an inverted condom when worn on the face, lips and chin, and can be worn over the penis and testicles. This prophylactic condom would provide an excellent defense against sexually transmitted diseases when engaging in sexual intercourse, oral or anal sex. The purpose to of the Multi-Purpose Face and Body Condom would be an exceptional preventive protector from body-to-body contact, genital warts, unwanted pregnancies, genital herpes, pubic lice and other sexually transmitted diseases.

### DESCRIPTION OF DRAWINGS

FIG. 1 shows frontal view of the Multi-Purpose Face and Body Condom with adjustable straps and plastic fastener.

FIG. 2 shows the Multi-Purpose Face and Body Condom with added feature of rubber hooking string attached to base of protective shield, which hooks onto the back of waistband for extra support during intercourse, mainly for the male user.

FIG. 3 shows the face and body condom with adjustable waistband fastened in a closed perspective view with attachable rubber string.

FIG. 4 show the face and body condom in a frontal view with the connected/circular waistband/headband.

FIG. 5 is a front view of an embodiment of the device being worn over the penis and testicles with rubber string attached to the hole in the shield where testicles are secured and adjustable waistband.

FIG. 6 shows another embodiment of the device of the backside of wearer with adjustable waistband, and the hole at base of condom with hooking rubber string between the buttocks attached to back of waistband.

FIG. 7 show side view of an embodiment of the device covering the male's penis and testicles with attached hooking rubber string between the buttocks attached to adjustable waistband.

FIG. 8 is a view of the reinforced hooking rubber string which hooks at the base of the protective shield of condom which goes between the buttocks and hooks to waistband.

FIG. 9 shows full front view of the device on face of a male.

FIG. 10 show a frontal view of an embodiment of the device when inverted on the male.

FIG. 11 show side view of the condom when inverted and worn by a male.

FIG. 12 shows back of the head view with adjustable straps worn on a male.

FIG. 13 show a frontal view of an embodiment of the device worn on a female.

FIG. 14 show a frontal view of the device worn by a female in an inverted manner.

FIG. 15 show the connected/circular headband on the back of the head.

### DETAILED DESCRIPTION OF INVENTION

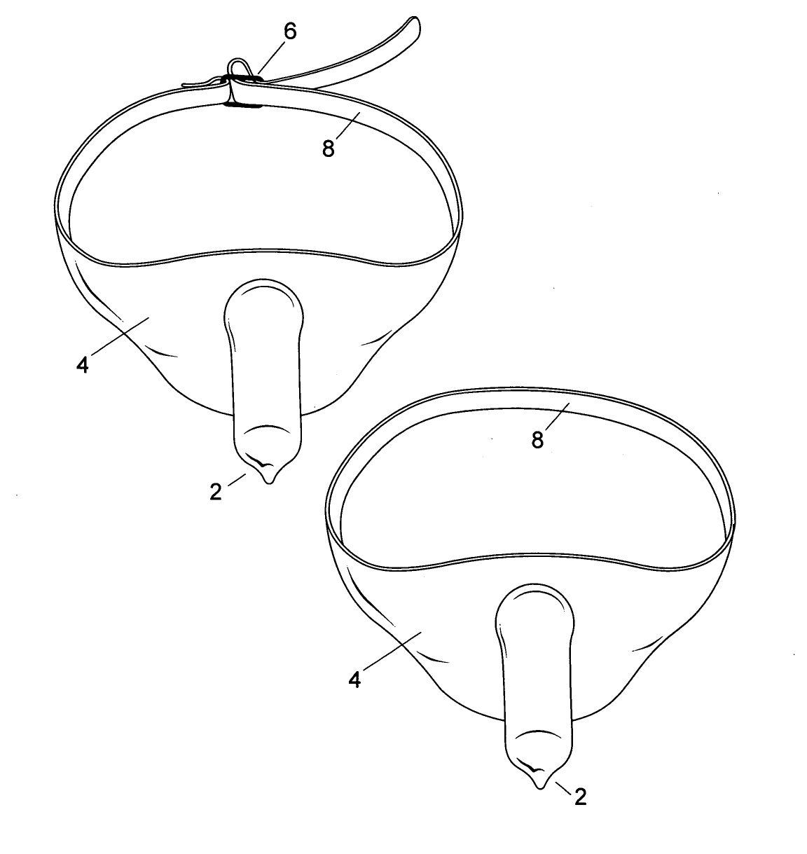

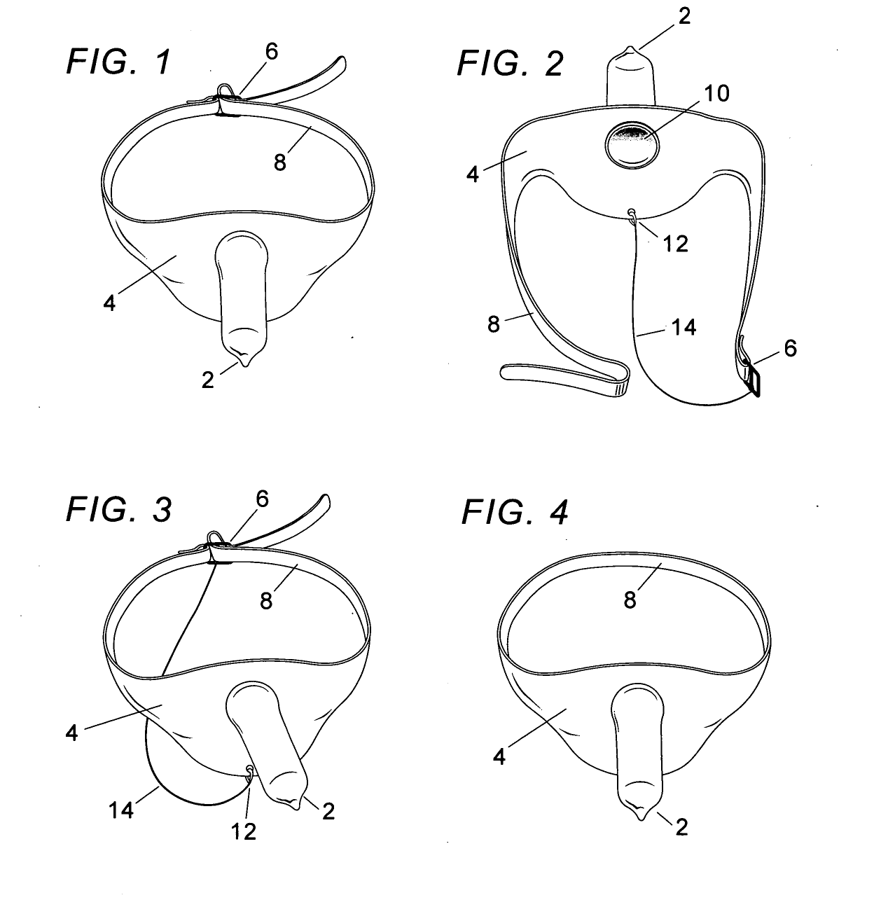

Several preferred embodiments of the invention are illustrated, as in FIG. 1 with facial shield protection, or it can be worn on the penis and testicles with adjustable waistband/headband with plastic adjustable fastener and in FIG. 4 show the invention with connected/circular waistband/headband to be worn on the face or worn on a man's lower body.

Referring to FIGS. 2 and 3 is specifically geared towards the male with looping rubber string to go between the buttocks with adjustable waistband. Although, this condom can also be worn by females in this manner if so desired.

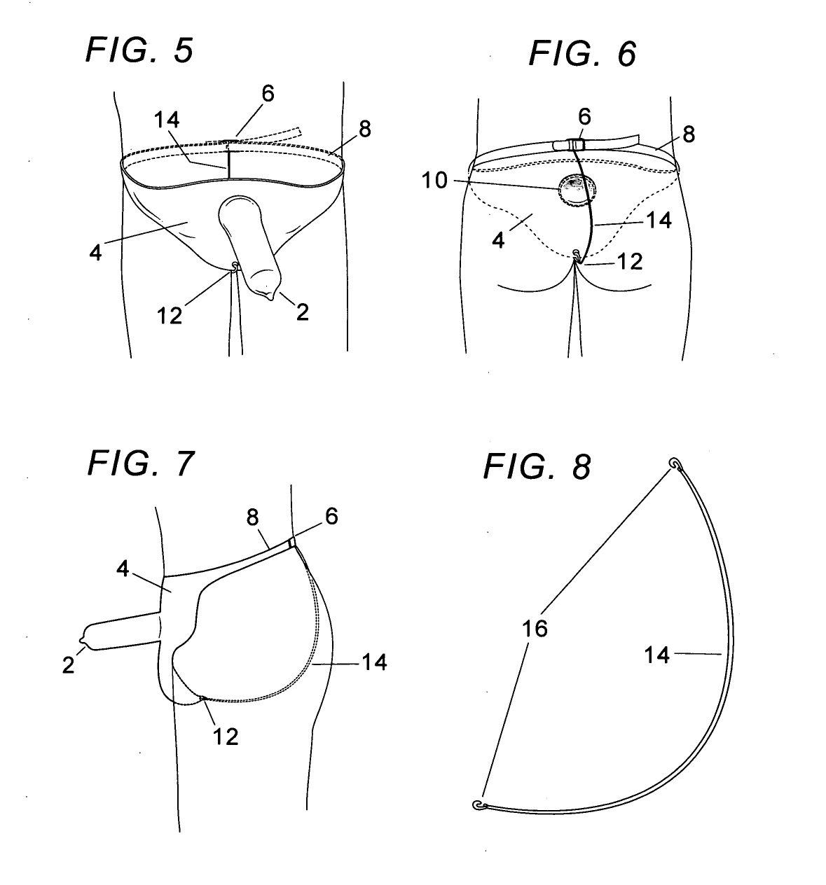

Referring to FIGS. 5,6 and 7 shows in 5 the body condom from the front view covering the penis and testicles with rubber string attached to the shield and waistband to hold the penis and testicles more secure in place from flouncing. FIG. 6 show the backside of the male with condom on with attaching rubber string and adjustable waistband for intercourse, anal sex or to receive oral sex. FIG. 7 show the side view of a male wearing the body condom with attached rubber hooking string attached to waistband.

referring to FIG. 8 is showing the rubber string with reinforced rubber hooks which goes between the buttocks.

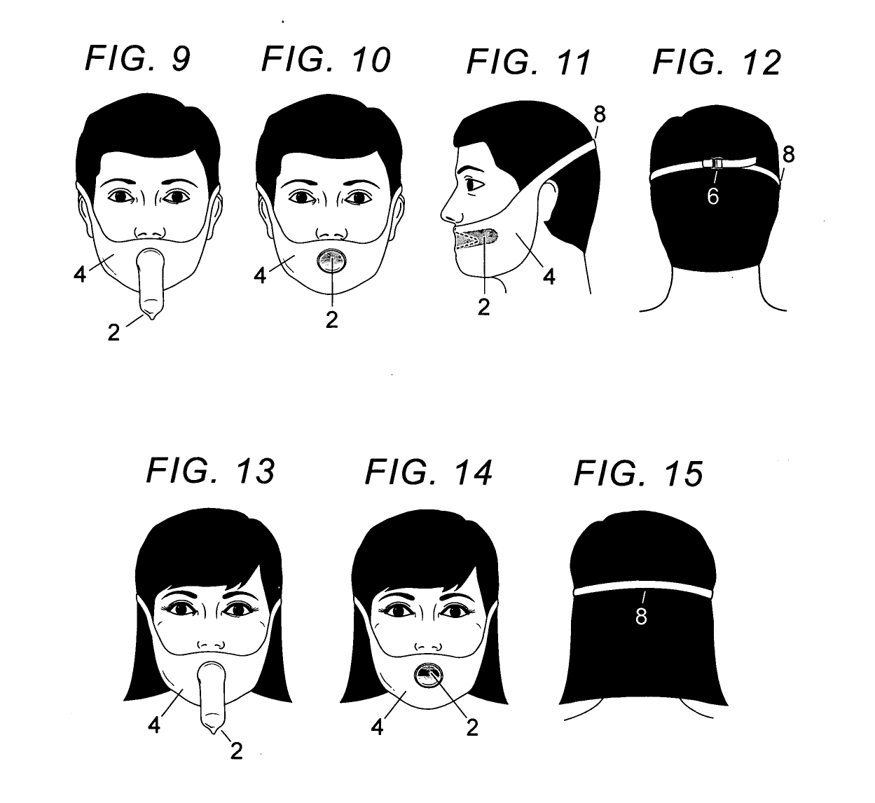

Referring to FIGS. 9,10,11 and 12, are examples of men wearing the body and face condom for protection of the face against sexually transmitted diseases. FIG. 9 show the male's front view of the facial shield protective condom positioned to give oral sex. FIG. 10 is a view of the male wearing the facial condom in the inverted manner, also, 11 shows the male wearer's side view of the inverted facial condom with band fitting on the back of the head, and FIG. 12 show the male wearer wearing the adjustable headband. Yet, either sex can wear the adjustable headband or the connected headband.

Referring to FIGS. 13,14 and 15 are all drawings of a female wearing the face condom on the frontal view of the face as in 13 the view is in a position to give oral sex to female or anal sex, 14 is a front facial view of inverted condom positioned to receive the male's penis. And FIG. 15 shows back of the head of female wearing connected headband. Either sex can wear the adjustable or connected headband/waistband.

The Multi-Purpose Face and Body Condom can be manufactured in many sizes ranging from small to extra, extra large and etc., in order to be able to fit comfortably on the lower face or lower body. There are many advantages to this condom: (a) The Multi-Purpose Face and Body Condom provides an effective barrier of protection against STD's for the wearer and their partner engaging in sexual intercourse, oral or anal sex. (b) The face and body condom is easy to wear, either by slipping the condom on the lower face positioned over the mouth and chin, and over the head, or with the adjustable straps inserted into the plastic adjustable fastener around the head. (c) One of the unique aspects of this condom is the circular shaped shield, which will completely encompass the wearer's testicles when worn over the penis to prevent body-to-body contact. (d) Another advantage to this condom is that when worn over the lower face, the condom can be inverted. (e) Another added advantage for the wearer indulging in sexual intercourse or anal sex is the rubber string. Some of the condoms can be made with a hole in the base of the condom shield so that the rubber string can hook on to the protective shield and fit between the buttocks and hook onto the back of the waistband to secure penis and testicles in place. (f) This protective face and body condom would protect users from unwanted pregnancy. (g) By purchasing a size larger for the face, or the body the user should be comfortable using this condom. (h) When used as a body condom over the penis and testicles preventing body-to-body contact will not only prevent the transmission of STD's, will also prevent the transmission of genital warts, genital herpes and pubic lice. (i) This Multi-Purpose Face and Body Condom could provide a comfortable alternative to ordinary condoms presently on the market. (j) Additional considerations could include the manufacture of lubricated or ribbed versions of the Multi-Purpose Face and Body Condom. (k) As with standard condoms, this condom would be made cylindrical in shape, manufactured of lightweight yet reliable latex or rubber materials, the adjustable headband/waistband fastener will be plastic. The tubular part of the condom would feature a durable, softly rounded reservoir for use in collecting bodily fluids. The reinforced band at the base of the unit would be designed to secure the condom in place.

What I claim as my invention is:

1. A Multi-Purpose Face and Body Condom comprising:
1. Of latex in material, cylindrical in shape with protective shield for chin and testicles, reinforced headband/waistband, with or without adjustable band, plastic adjustable fastener, durable, softly rounded tubular reservoir to collect bodily fluids, or protect said user of oral sex from diseases, a distinguishing feature is the banding which encompasses the base of the condom that extends outwards creating a circular shaped shield which would completely encompass said wearer's testicles when worn over the penis, as well as the said wearer's lips and chin when worn over the mouth. Can invert. Extra added feature on some condoms, a hole at base of condom shield for hooking rubber string to go between the buttocks to hook onto the waistband of the Multi-Purpose Face and Body Condom.

2. Face and body prophylactic as defined in claim 1 wherein, said membrane comprises of reliable latex.

3. The protective face and body condom as defined in claim 1 wherein, said waistband/headband is a reinforced band positioned at base of unit to secure condom in place.

4. The Multi-Purpose Face and Body Condom as defined in claim 1 wherein, said headband/waistband either solid or connecting with adjustable plastic fastener.

5. As pointed out in claim 1, said tubular part of condom can be inverted.

6. The protective device according to claim 1 wherein said circular shaped shield would completely encompass said wearer's penis and testicles.

7. As in claim 1 and 6, an oral prophylactic condom for use on lower face encompassing lips and chin, can be inverted on said user.

8. The protective body condom as defined in claim 1 and 7 wherein, said circular shaped shield could include a hole in base of shield for inserting a strong hooking rubber string from base of protective shield between said user's buttocks to hook onto said user's waistband.
